(19) United States
(12) Patent Application Publication
(10) Pub. No.: US 2023/0375536 A1
(43) Pub. Date: Nov. 23, 2023

(54) METHODS FOR PROXIMAL MOLECULAR PROBE TRANSFER

(71) Applicant: THE REGENTS OF THE UNIVERSITY OF CALIFORNIA, Oakland, CA (US)

(72) Inventors: Guillaume Castillon, La Jolla, CA (US); Stephen Adams, Poway, CA (US); Mark Ellisman, Solana Beach, CA (US); Daniela Boassa, Valley Center, CA (US)

(21) Appl. No.: 18/197,490

(22) Filed: May 15, 2023

Related U.S. Application Data
(60) Provisional application No. 63/343,469, filed on May 18, 2022.

(51) Int. Cl. G01N 33/533

(52) U.S. Cl. CPC G01N 33/533

(57) ABSTRACT

Methods and compositions provide for transfer of a detectable label, such as a photosensitizer, directly from a polypeptide of interest to an acceptor target molecule located in close proximity to the polypeptide of interest, facilitating detection of the target molecule. A multifunctional conjugation reagent is configured to provide such transfer.

### RELATED APPLICATIONS

This application claims the benefit of the priority of Provisional Application No. 63/343,469, filed May 18, 2022, which is incorporated herein by reference in its entirety.

### GOVERNMENT RIGHTS

This invention was made with government support under Grants R01 GM086197, 1S10OD021784, U24NS120055, R24GM137200, 5P41GM103412 and R01 GM086197 awarded by the National Institutes of Health. The government has certain rights in the invention.

### TECHNICAL FIELD

The present disclosure generally relates to methods and compositions for detection of biomolecules interacting with proteins and, in particular, to methods and related kits for biomolecule labeling.

### BACKGROUND

Most biological functions rely on interactions between proteins and a large variety of macromolecules such as other proteins, ribonucleotides, lipids, polysaccharides, and their metabolic intermediates. Through interactions, proteins can transform, concentrate, or segregate macromolecules and metabolites. Alternatively, macromolecules and metabolites are able to not only activate or inhibit the protein activities, but also influence their localization. For these reasons, it is important to identify what fraction of a protein is associated with a macromolecule, as it might reveal whether the protein is active or inactive. The same reasoning applies to metabolites. For example, when considering mRNAs, it is important to be able to distinguish whether they are transcribed, edited, translated, or degraded.

A method for capturing protein/metabolite interaction events based on light and electron microscopy would be particularly useful for analyzing protein/RNA interactions. For visualization by correlated light and electron microscopy (CLEM), the method should produce fluorescence and diffract electrons locally. Several approaches are known for studying protein-protein, protein-macromolecule or protein-metabolite interactions. One approach involves oxidizing diaminobenzidine (DAB) with a photosensitizer locally (see Maranto, A., Science, 217, September 1982). Upon illumination, fluorescent photosensitizers generate reactive singlet oxygen that oxidizes DAB in their near vicinity, triggering electron-dense polymerization. This approach has been used to detect Histone H2B via Halotag technology (see T. C. Binns, et al., Cell Chem. Biol. 27, 2020) Various metabolites, including DNA and RNA, can be directly labeled by the photosensitizers using click chemistry (J. T. Ngo, et al., Nat Chem Biol. 12(6), 2016). In another example, interactions can be visualized by Förster (or Fluorescence) Resonance Energy Transfer (FRET), as described by S. Ishiwata, et al. in Biophys J. 73, August 1997. Nonetheless, there remains a need in the art for improved techniques relating to detection of biomolecule interactions with proteins.

### SUMMARY

Disclosed herein are embodiments of an inventive approach of using light and electron microscopy compatible methods, as well as corresponding reagents, for capturing protein-macromolecule/metabolite interaction events. These and other embodiments of the invention will be apparent upon reference to the following detailed description. To this end, various references are set forth herein which describe in more detail certain background information, procedures, compounds and/or compositions, and are each hereby incorporated by reference in their entireties.

The inventive approach, referred to as “PROMPT”, for “PROximal Molecular Probe Transfer,” provides methods for labeling biomolecules, biological macromolecules, or small molecules that interact with a polypeptide, or are located in proximity to a polypeptide. With the analogy to FRET, which includes transferring energy from a donor fluorophore to an acceptor fluorophore, the inventive methods involve transferring a detectable label (e.g., fluorophore or fluorescent photosensitizer) directly from the polypeptide of interest to an acceptor target molecule (e.g., biological macromolecule, metabolite, small molecule inhibitor, etc.) located in close proximity to the polypeptide of interest, followed by detection of the target molecule. In some embodiments, detection of the target molecule is achieved using light or electron microscopy. Close physical proximity of the polypeptide and target molecule required for detectable label transfer ensures only interacting molecules are marked, while untransferred labels are removed before detection (e.g., imaging). To ensure high selectivity of the disclosed methods, removal of interfering background signals that obscure the interaction signal is necessary.

Some aspects of the invention include a multifunctional conjugation reagent, which comprises a first bioorthogonal reactive handle configured to be attached to a target molecule or to a modified target molecule; a detectable label; a second reactive handle configured to be attached to a polypeptide or to a modified polypeptide; and a linker located between the second reactive handle and the detectable label, wherein the linker comprises a selectively cleavable linkage.

The first bioorthogonal reactive handle may be selected from the group consisting of: azide, tetrazine, methyltetrazine, cyclopropene, trans-cyclooctene, substituted trans-cyclooctene, alkene, terminal alkyne, cyclooctyne tetrazine, ester, thioester, nitrile, alkylating agent, phosphate ester, azidoacetamide, semicarbazide, phospholipid, ketone, aldehyde, hydrazide, alkoxyamine, phosphine, nitrone, nitrile oxide, diazo compound, tetrazole, quadrocyclane, iodobenzene, cyclooctyne, bicyclononyne (BCN), diarylcyclooctyne (DBCO), norbornene, vinyl, isonitrile, and cycloaddition reactant. The second reactive handle may be a bioorthogonal reactive handle and may be selected from the group consisting of: a Halotag ligand, a SNAP ligand, a CLIP ligand, tetracysteine ligand, and a THP-ligand. The detectable label may include a fluorogenic moiety or a photosensitizer. In some embodiments, the detectable label may include biotin or a biotin derivative.

The selectively cleavable linkage may be selected from the group consisting of: a disulfide cleavable by reduction, a photolabile linkage or vicinal diol-containing linkage cleavable by periodate, and protease cleavable linkage.

In some embodiments, the target molecule may be a biological macromolecule selected from the group consisting of: a polynucleotide, a lipid, a target polypeptide, and a carbohydrate. In some embodiments, the target molecule is a first protein and the polypeptide is a second protein. The linker may further include a poly(ethylene glycol) PEG polymer, a poly(ethylene oxide) PEO polymer, a polymethylene, or a peptide.

In some embodiments, the first bioorthogonal reactive handle and the second reactive handle may be located at different termini of the conjugation reagent.

In another aspect of the invention, a method for labeling a target molecule that is located in proximity to a polypeptide includes the steps of: (a) providing the target molecule having a first complementary bioorthogonal reactive handle configured to react with a first bioorthogonal reactive handle of a multifunctional conjugation reagent, wherein the multifunctional conjugation reagent comprises the first bioorthogonal reactive handle, a detectable label, a second reactive handle configured to be attached to the polypeptide, and a linker located between the second reactive handle and the detectable label, wherein the linker comprises a selectively cleavable linkage; (b) contacting the polypeptide with the multifunctional conjugation reagent, thereby generating the polypeptide comprising the first bioorthogonal reactive handle and detectable label; (c) providing conditions for reaction between the first complementary bioorthogonal reactive handle of the target molecule and the first bioorthogonal reactive handle of the polypeptide; and (d) providing conditions for cleavage of the selectively cleavable linkage, wherein after the cleavage the detectable label remains attached to the target molecule. In some embodiments, the target molecule may specifically binds to the polypeptide.

When the method is used for labeling the target molecule located inside a cell, the labeling of the target molecule at step a) may occur within the cell, and the target molecule is in proximity to the polypeptide at step b) within the cell. The method may further include providing a fixation solution to the cell after step b) and before step c).

In some embodiments the first bioorthogonal reactive handle may be selected from the group consisting of: azide, tetrazine, methyltetrazine, cyclopropene, trans-cyclooctene, substituted trans-cyclooctene, alkene, terminal alkyne, cyclooctyne tetrazine, ester, thioester, nitrile, alkylating agent, phosphate ester, azidoacetamide, semicarbazide, phospholipid, ketone, aldehyde, hydrazide, alkoxyamine, phosphine, nitrone, nitrile oxide, diazo compound, tetrazole, quadrocyclane, iodobenzene, cyclooctyne, bicyclononyne (BCN), diarylcyclooctyne (DBCO), norbornene, vinyl, isonitrile, and cycloaddition reactant. The second reactive handle may be a bioorthogonal reactive handle. In some embodiments, the target molecule may be a first protein and the polypeptide may be a second protein.

The second reactive handle may be selected from the group consisting of: a Halotag ligand, a SNAP ligand, a CLIP ligand, tetracysteine ligand, and a THP-ligand. The detectable label may include a fluorogenic moiety or a photosensitizer, and/or may include biotin or a biotin derivative.

The selectively cleavable linkage may be selected from the group consisting of a disulfide cleavable by reduction, a photolabile linkage or vicinal diol-containing linkage cleavable by periodate, and protease cleavable linkage.

The target molecule may be a biological macromolecule selected from the group consisting of a polynucleotide, a lipid, a target polypeptide, and a carbohydrate.

The linker may further include a poly(ethylene glycol) PEG polymer, a poly(ethylene oxide) PEO polymer, a polymethylene, or a peptide. In some embodiments, the first bioorthogonal reactive handle and the second reactive handle are located at different termini of the conjugation reagent.

Other aspects of the inventive approach include methods for labeling a target molecule that is located in proximity to a polypeptide, the method comprising the steps of: (a) providing the target molecule having a first complementary bioorthogonal reactive handle configured to react with a first bioorthogonal reactive handle of a multifunctional conjugation reagent, wherein the multifunctional conjugation reagent comprises the first bioorthogonal reactive handle, a detectable label, a second reactive handle configured to be attached to the polypeptide, and a linker located between the second reactive handle and the detectable label, wherein the linker comprises a selectively cleavable linkage; (b) contacting the polypeptide with the multifunctional conjugation reagent, thereby generating the polypeptide comprising the first bioorthogonal reactive handle and detectable label; (c) providing conditions for reaction between the first complementary bioorthogonal reactive handle of the target molecule and the first bioorthogonal reactive handle of the polypeptide; and (d) providing conditions for cleavage of the selectively cleavable linkage, wherein after the cleavage the detectable label remains attached to the target molecule.

These and other features, aspects and advantages of the present teachings will become better understood with reference to the following description, examples, and appended claims.

### BRIEF DESCRIPTION OF THE DRAWINGS

Those of skill in the art will understand that the drawings, described below, are for illustrative purposes only. The drawings are not intended to limit the scope of the present teachings in any way.

FIGS. 1A-1C illustrate components of the PROMPT principles, where FIG. 1A provides an exemplary generic structure of multifunctional conjugation reagent (MCR, also called “PS-PROMPT”); FIG. 1B illustrates an exemplary structure of MCR used in Examples 1-6; and FIG. 1C diagrammatically depicts exemplary detectable labels (fluorescent photosensitizers) that were present in the MCR used in Examples 1-6.

FIG. 2 shows exemplary PROMPT method steps as described in Example 3.

FIG. 3 is an exemplary flowchart of the PROMPT labeling method.

FIG. 4 illustrates exemplary fluorescent photosensitizer spectra used in the PROMPT labeling method described in Examples 1-6.

FIG. 5 plots the average percentage of cells positive for JF570-PROMPT fluorescent signal after the PROMPT method as described in Example 3.

FIG. 6 plots results of an exemplary use of the PROMPT method for distance estimation in cells treated as described in Example 5.

FIG. 7 illustrates an exemplary use of Fibrillarin/RNA PROMPT CLEM method as described in Example 6.

FIG. 8 shows exemplary synthesis of TMR-PROMPT and TMR-PEG4-PROMPT probes. Carboxytetramethylrhodamine is a mixture of 5- and 6-isomers; only one isomer is shown for clarity.

FIG. 9 shows exemplary synthesis of JF525-PROMPT and JF570-PROMPT probes.

FIG. 10 shows exemplary reactions between target molecule (e.g., protein) modified with the first complementary bioorthogonal reactive handle (SNAP, CLIP or Halo polypeptides) and a first bioorthogonal reactive handle of a multifunctional conjugation reagent.

### DETAILED DESCRIPTION OF EMBODIMENTS

Numerous specific details are set forth in the following description in order to provide a thorough understanding of the present disclosure. These details are provided for the purpose of example and the claimed subject matter may be practiced according to the claims without some or all of these specific details. It is to be understood that other embodiments can be used and structural changes can be made without departing from the scope of the claimed subject matter, and that various features and functionality described in one or more of the individual embodiments are not limited in their applicability to the particular embodiment with which they are described.

### Definitions

Unless defined otherwise, all technical and scientific terms used herein have the same meaning as is commonly understood by one of ordinary skill in the art to which the present disclosure belongs. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, the singular forms “a,” “an” and “the” include plural referents unless the context clearly dictates otherwise. Thus, for example, reference to “a peptide” includes one or more peptides, or mixtures of peptides. Also, and unless specifically stated or obvious from context, as used herein, the term “or” is understood to be inclusive and covers both “or” and “and”.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X.

As used herein, the term “detectable label” refers to a substance which can indicate the presence of another substance when associated with it. The detectable label can be a substance that is linked to or incorporated into the substance to be detected. In some embodiments, a detectable label is suitable for allowing for detection and also quantification, for example, a detectable label that emits a detectable and measurable signal. Detectable labels include any labels that can be utilized and are compatible with the provided peptide analysis assay format and include, but not limited to, a bioluminescent label, a biotin/avidin label, a chemiluminescent label, a chromophore, a coenzyme, a dye, an electro-active group, an electrochemiluminescent label, an enzymatic label (e.g. alkaline phosphatase, luciferase or horseradish peroxidase), a fluorescent label, a magnetic particle, a metal, a metal chelate, a phosphorescent dye, a radioactive element or moiety, and a stable radical.

Examples of detectable labels especially useful for methods and compositions described herein include, but are not limited to, tetramethyl rhodamine, rhodamine B, 5-Carboxytetramethylrhodamine (5-TAMRA), 1,4,7,10-Tetraazacyclododecane-1,4,7,10-tetraacetic acid (DOTA), Janelia Fluors 525 and 570, dibromofluorescein, eosin, IRDye 700DX, fluorescein, (aza)bodipy, methylene blue, cyanines, desthiobiotin, and any small molecule fluorophore suitable for a fluorescent readout.

The term “fluorogenic moiety”, as used herein, refers to a moiety that contributes to generation of a fluorescent signal that can be detected. Fluorogenic moieties include fluorescent groups, such as fluorescent dyes disclosed herein, fluorescence quenchers, and combination of these. In some embodiments, a fluorogenic moiety comprises a fluorophore proximal to a moiety that interacts through bonds or through space with the fluorophore, such as a quencher. In these embodiments, the presence of or changes in the fluorophore can be used to monitor the progress of reactions used to modify a target molecule or to link a first target molecule to a second target molecule.

As used herein, the term “reactive handle” refers to a moiety on a first molecule that can be caused to react with a second molecule having a complementary ‘reactive handle’ to form a covalent bond between the first molecule and the second molecule. Typical reactive handles include functional groups such as carboxylate groups and amines, which can react with each other to form amides; thiols and alkylating reagents that can be reacted to form thioethers; thiols and maleimides that can be reacted to form thiosuccinimides; strained alkenes or alkynes and 1,3-dipoles such as azides that can react via cycloaddition reactions, e.g., copper-free click chemistry; and tetrazines that can react via inverse-electron demand Diels-Alder chemistry with electron rich or strained alkenes and alkynes.

“Bioorthogonal” reactive handles are reactive handles that can be used in biological systems, i.e., in aqueous media, and that are generally not reactive toward common functional groups in the biological system, so they can be used to manipulate biological compounds selectively, without interference from the biomolecule components. Bioorthogonal chemistry is well known in the art: suitable functional groups for bioorthogonal chemistry include ketones, aldehydes, hydrazides, alkoxyamines, azides, terminal alkynes, phosphines, nitrones, nitrile oxides, diazo compounds, tetrazines, tetrazoles, quadrocyclanes, alkenes, iodobenzenes, transcyclooctenes, cyclooctynes, norbornenes, cyclopropenes, vinyls, isonitriles, and cycloaddition reactants. M. F. Debets, et al., Org. Biomol. Chem. 2013, vol. 11, 6439. Examples include click chemistry, particularly copper-free click chemistry, which uses cycloaddition reactants like cyclooctyne that react efficiently with alkyl azides; and inverse-electron demand Diels-Alder chemistries such as tetrazines, which react with strained alkenes or alkynes like cyclopropene and trans-cyclooctene as well as strained alkynes like cyclooctynes. Useful cyclooctynes include:

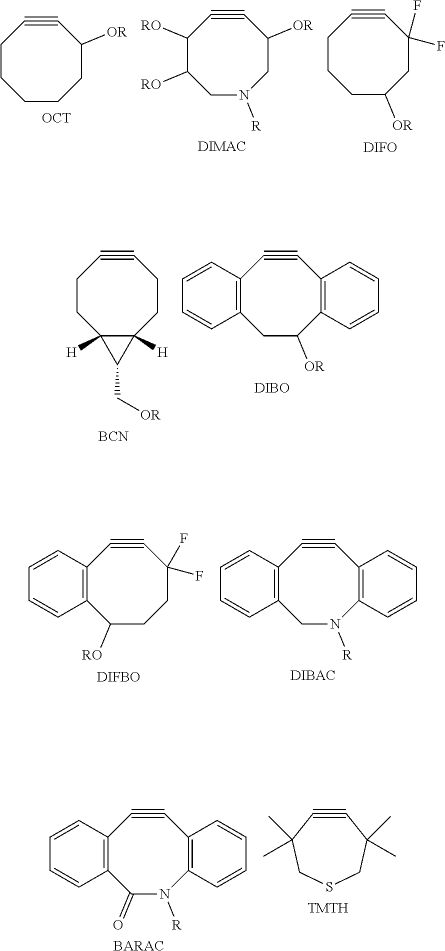

‘R’ in these structures indicates where the cyclooctyne compound can be attached to a target molecule or conjugation reagent, etc. TMTH is actually a 7-membered ring, but the C—S bonds are longer than C—C bonds, so the ring strain is similar to that of a cyclooctyne. (See, e.g., C. P. Ramil, et al., Chem. Commun. 2013, vol. 49, 11007-11022.)

As used herein, “click chemistry” refers to reactions and reactants that are commonly used in biological systems and are useful as reactive handles in the conjugation reagents and methods according to embodiments of the invention. Click chemistry reactive handles include reactants for inverse-electron demand Diels-Alder reactions, such as tetrazines, which react efficiently with a variety of activated alkene and alkyne groups such as cyclopropenes and trans-cyclooctene, and reactants for [3+2] cycloadditions, such as azide which reacts efficiently with an electron rich alkene or alkyne. Tetrazines are well known reactive handles for attaching fluorogenic probes to biomolecules such as peptides to enable visualization of target biomolecules in cells. (See, e.g., Y. Lee, et al., J. Am. Chem. Soc. 2018, 140, 974-983.) Tetrazine rings are stable in biological media, and react with specific reaction partners under mild conditions, so they are very useful for attaching a probe to a target with good selectivity.

As used herein, the term “sample” refers to anything which may contain an analyte for which an analyte assay is desired. As used herein, a “sample” can be a solution, a suspension, liquid, powder, a paste, aqueous, non-aqueous or any combination thereof. The sample may be a biological sample, such as a biological fluid or a biological tissue. Examples of biological fluids include urine, blood, plasma, serum, saliva, and others. Biological tissues are aggregate of cells, usually of a particular kind together with their intercellular substance that form one of the structural materials of a human, animal, plant, bacterial, fungal or viral structure, including connective, epithelium, muscle and nerve tissues. As used herein, a “biological sample” refers to any sample obtained from a living or viral source of macromolecules and biomolecules, and includes any cell type or tissue of a subject from which nucleic acid, protein and/or other biological macromolecules can be obtained. The term “subject” includes a mammal. The biological sample can be a sample obtained directly from a biological source or a sample that is processed.

The terms “level” or “levels” are used to refer to the presence and/or amount of a target, e.g., a substance or an organism that can be determined qualitatively or quantitatively. A “qualitative” change in the target level refers to the appearance or disappearance of a target that is not detectable or is present in samples obtained from normal controls. A “quantitative” change in the levels of one or more targets refers to a measurable increase or decrease in the target levels when compared to a normal control.

As used herein, the term “macromolecule” encompasses large molecules composed of smaller subunits. Examples of macromolecules include, but are not limited to peptides, nucleic acids, carbohydrates, lipids, macrocycles, or a combination or complex thereof. A macromolecule also includes a chimeric macromolecule composed of a combination of two or more types of macromolecules, covalently linked together (e.g., a peptide linked to a nucleic acid). A macromolecule assembly may be composed of the same type of macromolecule (e.g., protein-protein) or of two or more different types of macromolecules (e.g., protein-DNA).

As used herein, the term “target molecule” refers to any molecule of interest that is located in a close proximity from a polypeptide of interest (POI), or binds to the polypeptide of interest, and can be detected using a detectable label after transferring the detectable label from the polypeptide of interest. A target molecule can be a biological macromolecule such as a polynucleotide, a lipid, a polypeptide, or a carbohydrate. A target molecule can be a small molecule, such as a small molecule modulator, which specifically binds to the polypeptide of interest. The proximity (distance) between POI and a molecule of interest (target molecule) should be no more than a length of the multifunctional conjugation reagent utilized in the described proximal molecular probe transfer assay. In preferred embodiments, maximal distance between POI and a molecule of interest is determined by and can be controlled by the linker located between the second reactive handle and the detectable label of the multifunctional conjugation reagent. In preferred embodiments, maximal distance (proximity) between POI and a target molecule for the disclosed methods is no more than 2 nm, 5 nm, 10 nm, 15 nm, 20 nm, 30 nm, 40 nm, 50 nm, 75 nm, or 100 nm.

The term “peptide” is used interchangeably with the term “polypeptide” and encompasses peptides and proteins, and refers to a molecule comprising a chain of two or more amino acids joined by peptide bonds. In some embodiments, a peptide comprises 3 to 50 amino acids. In some embodiments, a peptide does not comprise a secondary, tertiary, or higher structure. In some embodiments, the peptide is a protein. In some embodiments, a protein comprises 30 or more amino acids. In some embodiments, in addition to a primary structure, a protein comprises a secondary, tertiary, or higher structure. The amino acids of the peptides are most typically L-amino acids, but may also be D-amino acids, modified amino acids, amino acid analogs, amino acid mimetics, or any combination thereof. Peptides may be naturally occurring, synthetically produced, or recombinantly expressed. The term also encompasses an amino acid polymer that has been modified naturally or by intervention; for example, disulfide bond formation, glycosylation, lipidation, acetylation, phosphorylation, or any other manipulation or modification.

As used herein, the term “linker” refers to one or more of a nucleotide, a nucleotide analog, an amino acid, a peptide, a peptide, a polymer, or a non-nucleotide chemical moiety that is used to join two molecules. A linker may be used to join a binding agent with a coding tag, a recording tag with a peptide, a peptide with a support, a recording tag with a solid support, etc. In certain embodiments, a linker joins two molecules via enzymatic reaction or chemistry reaction (e.g., click chemistry).

The term “ligand” as used herein refers to any molecule or moiety connected to the compounds described herein. “Ligand” may refer to one or more ligands attached to a compound. In some embodiments, the ligand is a pendant group or binding site (e.g., the site to which the binding agent binds).

The term “modified” or “engineered” as used in reference to nucleic acid molecules, protein molecules, or target molecules, implies that such molecules are created by human intervention and/or they are non-naturally occurring. The modified target molecule has structural element(s) that the unmodified, original target molecule does not have, and which is/are created by addition of a moiety to the target molecule, by substitution of one component of the target molecule to another or a combination thereof.

The terms “specifically binding” and “specifically recognizing” are used interchangeably herein and generally refer to a polypeptide of interest that binds to a target molecule more readily than it would bind to a random, non-cognate molecule. The term “specificity” is used herein to qualify the relative affinity by which a polypeptide of interest binds to a cognate target molecule. Specific binding typically means that a polypeptide of interest binds to a cognate target molecule at least twice as likely as to a random, non-cognate molecule (a 2:1 ratio of specific to non-specific binding). In some embodiments, specific binding refers to binding between a polypeptide of interest and a target molecule with a dissociation constant (Kd) of 200 nM or less.

The methods disclosed herein include use of a multifunctional conjugation reagent (or MCR), which comprises a first bioorthogonal reactive handle configured to be attached to a target molecule or to a modified target molecule; a detectable label; a second reactive handle configured to be attached to a polypeptide or to a modified polypeptide; and a linker located between the second reactive handle and the detectable label, wherein the linker comprises a selectively cleavable linkage.

This MCR, which may also be referred to as “PS-PROMPT” for PhotoSensitizer-PROMPT, wherein “PROMPT” as used herein refers to a PROximal Molecular Probe Transfer, reflects the essence of the disclosed methods. PROMPT relies on a detectable label (e.g., a modified photosensitizer) transferred through sequential binding, from a polypeptide of interest to a proximal target molecule of choice.

In some aspects, the inventive approach includes methods for labeling a target molecule that is located in proximity to a polypeptide, the method comprising the steps of: (a) providing the target molecule having a first complementary bioorthogonal reactive handle configured to react with a first bioorthogonal reactive handle of a multifunctional conjugation reagent (MCR), wherein the multifunctional conjugation reagent comprises the first bioorthogonal reactive handle, a detectable label, a second reactive handle configured to be attached to the polypeptide, and a linker located between the second reactive handle and the detectable label, wherein the linker comprises a selectively cleavable linkage; (b) contacting the polypeptide with the multifunctional conjugation reagent, thereby generating the polypeptide comprising the first bioorthogonal reactive handle and detectable label; (c) providing conditions for reaction between the first complementary bioorthogonal reactive handle of the target molecule and the first bioorthogonal reactive handle of the polypeptide; and (d) providing conditions for cleavage of the selectively cleavable linkage, wherein after the cleavage the detectable label remains attached to the target molecule.

The first bioorthogonal reactive handle of the MCR may be selected from the group consisting of: azide, tetrazine, methyltetrazine, cyclopropene, trans-cyclooctene, substituted trans-cyclooctene, alkene, terminal alkyne, cyclooctyne tetrazine, ester, thioester, nitrile, alkylating agent, phosphate ester, azidoacetamide, semicarbazide, phospholipid, ketone, aldehyde, hydrazide, alkoxyamine, phosphine, nitrone, nitrile oxide, diazo compound, tetrazole, quadrocyclane, iodobenzene, cyclooctyne, bicyclononyne (BCN), diarylcyclooctyne (DBCO), norbornene, vinyl, isonitrile, and cycloaddition reactant.

The second reactive handle of the MCR may be a bioorthogonal reactive handle. In preferred embodiments, the second reactive handle is different from the first bioorthogonal reactive handle, so that the first bioorthogonal reactive handle of MCR is not configured to react with a peptide or peptide component, and the second reactive handle of MCR is not configured to react with a target molecule of choice. The second reactive handle may be selected from the group consisting of: a Halotag ligand, a SNAP ligand, a CLIP ligand, tetracysteine ligand, and a THP-ligand. In preferred embodiments, the detectable label may comprise a fluorogenic moiety or a photosensitizer. The detectable label may be biotin or a biotin derivative.

The selectively cleavable linkage may be one of the following: a disulfide cleavable by reduction, a photolabile linkage or vicinal diol-containing linkage cleavable by periodate, protease cleavable linkage. In other embodiments, other selectively cleavable linkages can be utilized, such as disclosed in Leriche, G., Chisholm, L., & Wagner, A. (2012). Cleavable linkers in chemical biology. Bioorganic & Medicinal Chemistry, 20(2), 571-582.

In some embodiments, the target molecule is a biological macromolecule, such as a polynucleotide, a lipid, a target polypeptide, or a carbohydrate. In some embodiments, the target molecule is a polynucleotide. In other embodiments, the target molecule is a target polypeptide. In some embodiments, the target molecule is not a lipid, not a target polypeptide, or not a carbohydrate. In other embodiments, the target molecule is not a polynucleotide. In yet other embodiments, the target molecule is a small molecule, for example, a small molecule inhibitor, or a small molecule modulater of an enzyme.

The linker may further include a poly(ethylene glycol) (PEG) polymer, a poly(ethylene oxide) (PEO) polymer, a polymethylene, or a peptide. Exemplary peptide linkers include flexible peptide linkers comprised of Gly or Ser residues.

The first bioorthogonal reactive handle and the second reactive handle may be located at different termini of the conjugation reagent.

In some embodiments, the target molecule specifically binds to the polypeptide. In other embodiments of the disclosed methods, the target molecule does not bind directly to the polypeptide, but rather is located in close proximity to the polypeptide. In these embodiments, the length of the linker of MCR determines whether the target molecule can be labeled by the disclosed methods. If the target molecule is located at a certain distance from the polypeptide, the length of the linker of MCR can be adjusted, so that the target molecule can be labeled by the disclosed methods. In some embodiments, the linker of MCR contains several subunits of a polymer, such as PEG polymer, or PEO polymer, and the number of subunits in the linker determines the length of the linker.

In some embodiments, the method can be used for labeling the target molecule located inside a cell, wherein labeling of the target molecule at step a) occurs within the cell, and the target molecule are provided in proximity to the polypeptide at step b) within the cell.

In preferred embodiments of the disclosed methods, the target molecule is modified (labeled) to incorporate a first complementary bioorthogonal reactive handle configured to react with a first bioorthogonal reactive handle of a multifunctional conjugation reagent. In other embodiments of the disclosed methods, the target molecule already contains a first complementary bioorthogonal reactive handle configured to react with a first bioorthogonal reactive handle of a multifunctional conjugation reagent, so no labeling step is required.

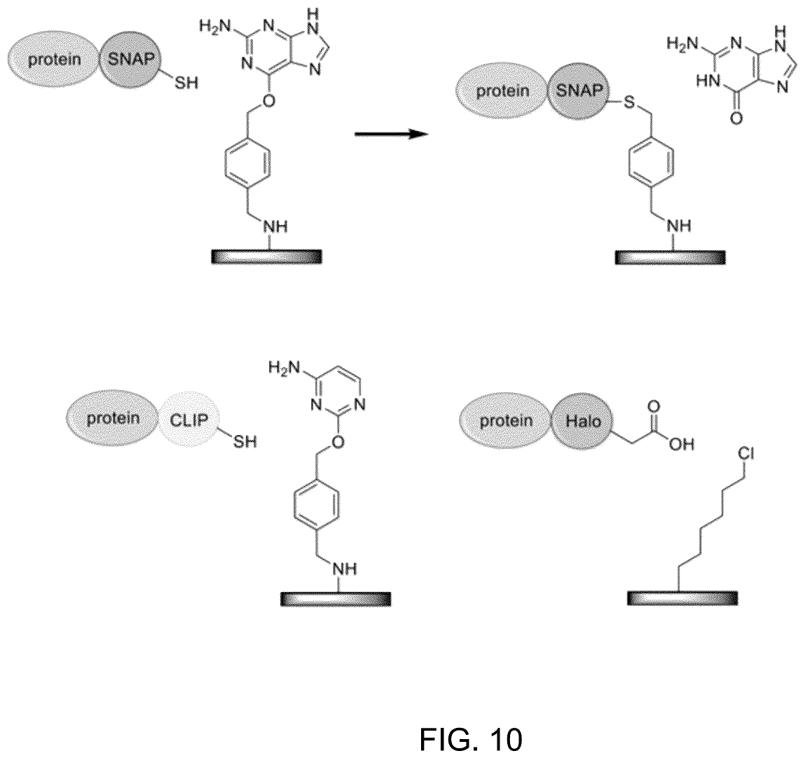

In some embodiments, the first complementary bioorthogonal reactive handle is selected from commercially available tagging substrates of the group consisting of Halotag polypeptide (GenBank Accession No. HM157289), a SNAP polypeptide (GenBank Accession No. AQS79239) and a CLIP polypeptide (GenBank Accession No. AQS79240), the latter two of which are part of the SNAP-Tag® technologies available from New England Biolabs, Inc. (Information within the NIH National Center for Biotechnology Information (NCBI.NLM.NIH) associated with the identified accession numbers are incorporated herein by reference in their entireties.) Exemplary reactions between target molecule modified with the first complementary bioorthogonal reactive handle and a first bioorthogonal reactive handle of a multifunctional conjugation reagent are shown in FIG. 10.

In some embodiments, the method further comprises providing a fixation solution to the cell after step b) and before step c).

In some embodiments, providing the target molecule having a first complementary bioorthogonal reactive handle configured to react with a first bioorthogonal reactive handle of a multifunctional conjugation reagent comprises labeling the target molecule having a first complementary bioorthogonal reactive handle configured to react with a first bioorthogonal reactive handle of a multifunctional conjugation reagent.

### EXAMPLES

Aspects of the present teachings may be further understood upon consideration of the following examples, which should not be construed as limiting the scope of the present teachings in any way.

### Example 1. Design and Synthesis of PS-PROMPT

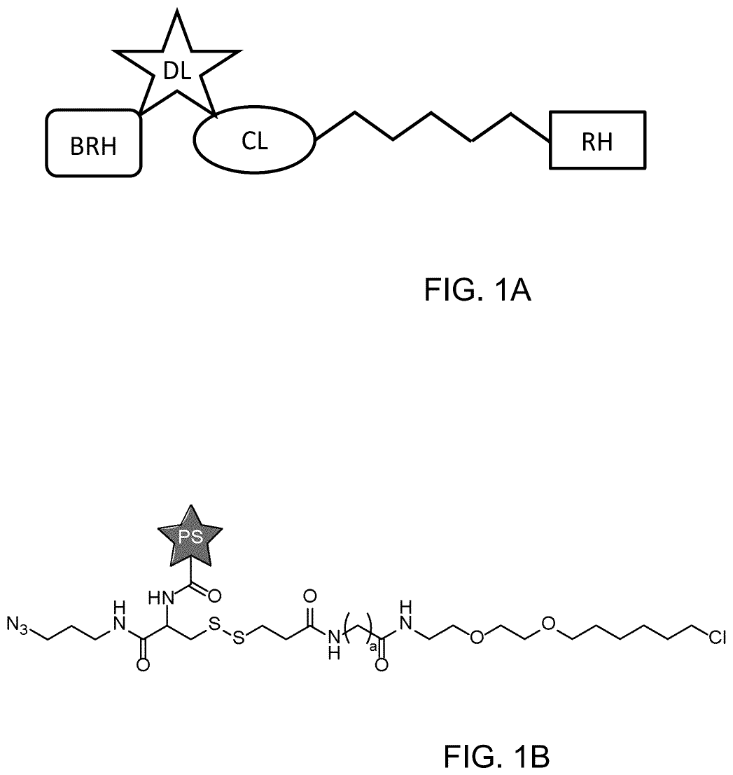

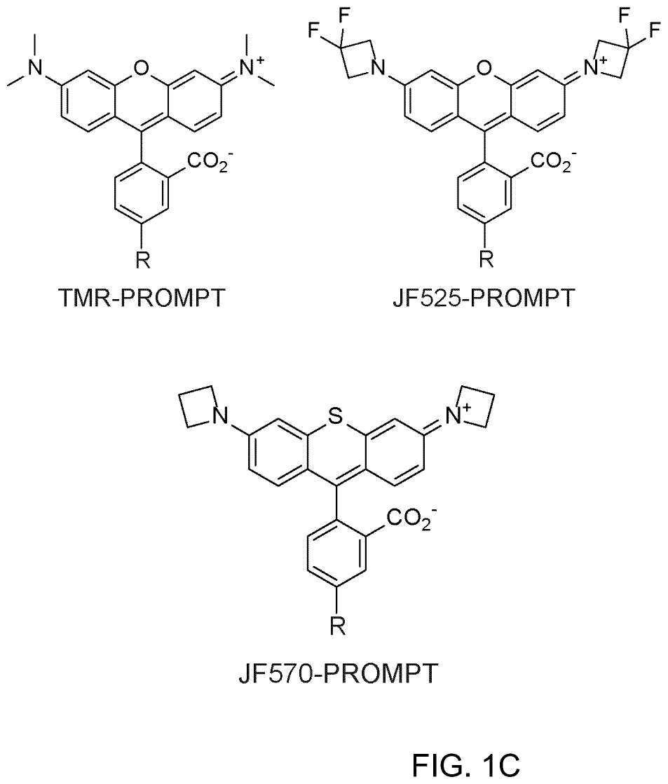

The labeling methods disclosed herein utilize a multifunctional conjugation reagent (MCR), generically named PS-PROMPT for PhotoSensitizer-PROMPT. Generic structure of PS-PROMPT is shown in FIG. 1A, where the components of the MCR: DL—detectable label; CL—cleavable linkage; BRH—first bioorthogonal reactive handle; and RH—second reactive handle. An exemplary PS-PROMPT is shown in FIG. 1B, in which the following components are labeled: N₃—azido group; PS—Photosensitizer; SS—disulfide bond; HTL—Halotag ligand, i.e., chloroalkane; L—linker having variable carbon or polyethyleneglycol chain length.

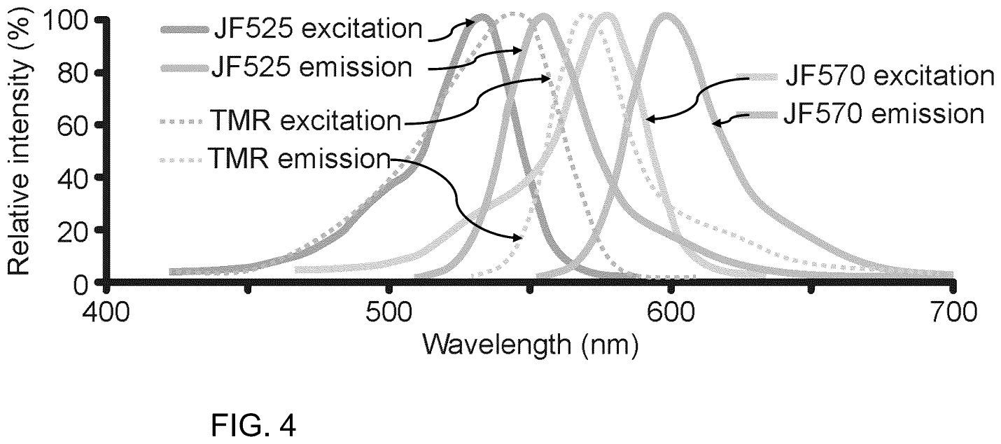

Specifically, the MCR includes a chloroalkane group and a Halotag ligand (HTL) as a second reactive handle configured to be attached to a polypeptide; an azido group (N₃) as a first bioorthogonal reactive handle configured to be attached to a target molecule or to a modified target molecule; a disulfide bond (S—S) that can be cleaved by reduction as a selectively cleavable linkage; and a fluorescent photosensitizer (PS) as a detectable label. Referring to FIG. 1C, for application to correlative light and electron microscopy approaches, the Janelia fluorophores, JF525 and JF570 can be chosen as potent photosensitizers (for DAB photooxidation). Alternatively, tetramethylrhodamine (TMR) can be chosen. The fluorescence emission peaks of these dyes range from 549-599 nm (see FIG. 4).

To test the functionality of each module of PS-PROMPT, TMR-PROMPT was synthesized (see below). 5(6)-carboxy-tetramethylrhodamine (TMR) was reacted with the 3-azidopropyl amide of S-trityl cysteine, prepared by reaction of Fmoc-NH-cys(S-Trt)-OH and azidopropylamine followed by removal of the N-terminal Fmoc. Cleavage of the trityl group and subsequent reaction with the SPDP conjugate of Halotag linker amine afforded TMR-PROMPT.

### Example 2. Synthesis of Key Elements of the Labeling Method (PROMPT)

Cell Culture, 5EU and EdU Labeling, Transfection.

U2OS cells were cultured on 35 mm MatTek dishes (MatTek Corp) in DMEM supplemented with 10% FBS at 5% CO2. When reaching 70% confluency and two days before the experiment, cells were transfected with 0.5 ug of DNA with lipofectamine 3000 (ThermoFisher Scientific) following the manufacturer protocol. If needed, cells were incubated overnight in culture medium with 5 uM of 5-Ethynyl 2′-deoxyuridine (EdU, #1149, click chemistry tools) diluted from a 10 mM stock in DMSO. For 5-EU (5-Ethynyl Uridine) incorporation, 200 mM 5-EU (#1261, click chemistry tools) in DMSO was diluted to 1 mM in culture medium and incubated on cells overnight.

DNA Constructs.

For the construct of Halotag-H2B (JH1348), the H2B sequence was amplified from pminiSOG-H2B-6 (Shu, X. et al. A Genetically Encoded Tag for Correlated Light and Electron Microscopy of Intact Cells, Tissues, and Organisms. PLoS Biol. 9, e1001041 (2011)) and substituted EGFP in pHaloTag-EGFP (addgene #86629) (Ebner, et al., PI(3,4,5)P3 Engagement Restricts Akt Activity to Cellular Membranes. Mol. Cell 65, 416-431.e6 (2017)) through the In-Fusion cloning method. For the construct of Halotag-Fibrillarin (JH1239), the Fibrillarin sequence was amplified from pEGFP-C1-Fibrillarin (addgene #26673) (Chen, D. & Huang, S. Nucleolar Components Involved in Ribosome Biogenesis Cycle between the Nucleolus and Nucleoplasm in Interphase Cells. The Journal of Cell Biology, vol. 153) and substituted EGFP in pHaloTag-EGFP (addgene #86629) through the In-Fusion cloning method. (Data and sequence listings for pEGFP-C1-Fibrillarin (Addgene plasmid #26673; http://n2t.net/addgene:26673; RRID:Addgene_26673) are available on the World Wide Web at addgene.org, which are incorporated herein by reference.)

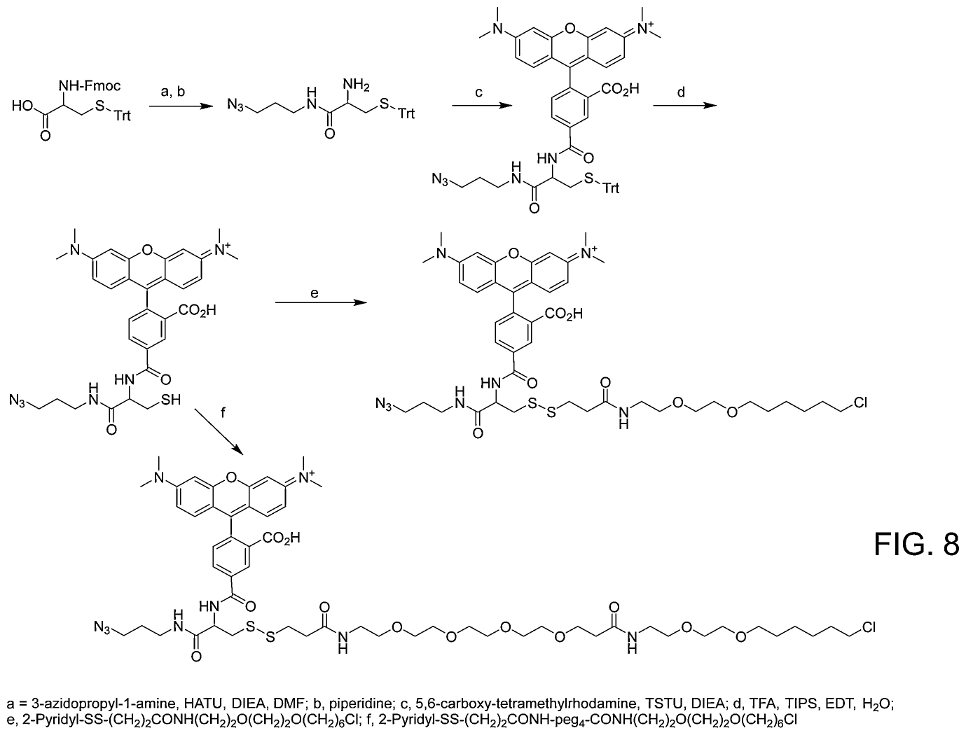

### Synthesis of TMR-PROMPT and TMR-PEG4-PROMPT (See Also FIG. 8)

### Synthesis of NH₂-cys(S-Trt)-CO—NH—(CH₂)₃—N₃

Fmoc-NH-cys(S-Trt)-OH (19.8 mg, 33.8 μmol) and HATU (14.1 mg, 37.2 μmol) were dissolved in dry DMF (100 μL) in a plastic screw cap tube and 3-azidopropylamine (3.7 L, 37.2 μmol, Click Chemistry Tools) followed by DIEA (13 μL, 74.42 μmol) were added with mixing. The reaction mix turned yellow and LC-MS revealed complete reaction in 30 min; ES-MS (m/z) [M+Na]⁺ for C₄₀H₃₇N₅NaO₃S, 690.25; found 689.3. Piperidine (20 μL, 0.2 mmol) was added and the solution evaporated under high vacuum after 1 h, dissolved in DMSO, separated by RP-HPLC and lyophilized to give a white solid. Yield, 14.3 mg, 76%. ES-MS (m/z) [M]+, [M+Na]⁺ for C₂₅H27N₅OS, 446.2, 468.2; found 446.1, 468.1.

### Synthesis of 5(6)-TMR-CONH-cys(SH)—CO—NH—(CH₂)₃—N₃

5(6)-Carboxytetramethylrhodamine, 5(6)-TMR-CO₂H (1.35 mg, 3.14 μmol, Novabiochem) and TSTU (1.3 mg, 4.4 μmol) were dissolved in dry DMSO (25 μL) with TEA (0.96 μL, 6.9 μmol) and kept at room temperature. Reaction was complete in 30 min (by LC-MS) and then added to a solution of NH₂-cys(S-Trt)-CO—NH—(CH₂)₃—N₃ (2.0 mg, 3.6 μmol) in DMSO (10 μL) with NMM (1 μL, 9.1 μmol) and kept at room temperature overnight when LC-MS revealed complete reaction. After acidification with HOAc (2 μL), the desired product was isolated by RP-HPLC and lyophilized to a red solid. Yield, 2.0 mg (74%) ES-MS (m/z) [M]⁺ for C₅₀H₄₈N₇O₅S, 858.3; found 858.3. The trityl group was removed by dissolving the product (1.89 mg, 2.2 μmol) in TFA:H₂O:Triisopropylsilane:Ethanedithiol (92.5:2.5:2.5:2.5 v/v, 0.5 mL) for 30 mins, evaporation under high vacuum, purification by RP-HPLC and lyophilization to a red solid. Yield, 0.9 mg (66%) ES-MS (m/z) [M]⁺ for C₃₁H₃₄N₇O₅S, 616.2; found 616.1.

### Synthesis of SPDP-HaloTag Linker

HaloTag linker amine (3.0 mg, 13.5 μmol) and SPDP (4.7 mg, 15 μmol) were dissolved in dry DMSO (50 μL) and NMM (3.3 μL, 30 μmol) was added. LC-MS revealed reaction was complete after overnight when the reaction mixture was neutralized with HOAc (5 μL) and the product was purified by RP-HLPC (and lyophilized to a colorless oil. Yield, 3 mg (54%) ES-MS (m/z) [M]⁺ for C₁₈H₃₀ClN₂O₃S₂, 421.1; found 421.1.

### Synthesis of TMR-PROMPT; 5(6)-TMR-CONH-cys(S—S—(CH₂)₂CONH-HaloTag ligand)-CO—NH—(CH₂)₃—N₃

A solution of 5(6)-TMR-CONH-cys(SH)—CO—NH—(CH₂)₃—N₃ in DMSO (100 μL, 6.25 mM measured by absorbance in 0.1 M HCl in 95% ethanol using εₘₐₓ 95000 M⁻¹ cm⁻¹ at 554 nm, 0.626 μmol) was mixed with SPDP-HaloTag linker (20 μL, 49 mM in dry DMSO, 0.98 μmol) and NMM (1 μl, 10 μmol) was added. After 1 h, HOAc (5 μL) was added and the desired product purified by RP-HPLC to give a colorless oil. Yield, 0.42 mg (72%) ES-MS (m/z) [M]⁺ for C₄₄H₅₈ClN₈O₈S₂, 925.4; found 925.4.

### Synthesis of SPDP-PEG4-HaloTag Linker

Solutions of HaloTag linker amine (3.0 mg, 4.5 μmol) in dry DMSO (90 μL) and SPDP-PEG4-NHS (2.5 mg, 4.5 μmol, Quanta Biodesign) in dry DMSO (90 μL) were mixed and NMM (1 μL, 10 μmol) was added. LC-MS revealed reaction was complete after 4 h and the product was used without further purification. ES-MS (m/z) [M]⁺ for C₂₉H₅₁ClN₃O₈S₂, 668.3; found 668.3.

### Synthesis of TMR-PEG4-PROMPT: 5(6)-TMR-CONH-cys(S—S—(CH₂)₂CONH-PEG4-HaloTag ligand)-CO—NH—(CH₂)₃—N₃

5(6)-TMR-CONH-cys(SH)—CO—NH—(CH₂)₃—N₃ (3 mL of 0.25 mM in DMSO; measured as above) was added to the solution of SPDP-PEG4-HaloTag Linker (15 μL of 50 mM, 0.75 μmol) and NMM (15 μl, 150 μmol) added. After 2 h, LC-MS indicated reaction was complete, HOAc (50 μL) was added, the product purified by RP-HPLC and lyophilized. Yield, 0.7 μmol (by absorbance in 0.1M HCl in 95% ethanol using Fmax 95000 M⁻¹ cm⁻¹ at 554 nm) after dissolving in dry DMSO (100 μL) ES-MS (m/z) [M]⁺ for C₅₅H₇₉ClN₉O₁₃S₂, 1172.5; found 1172.4.

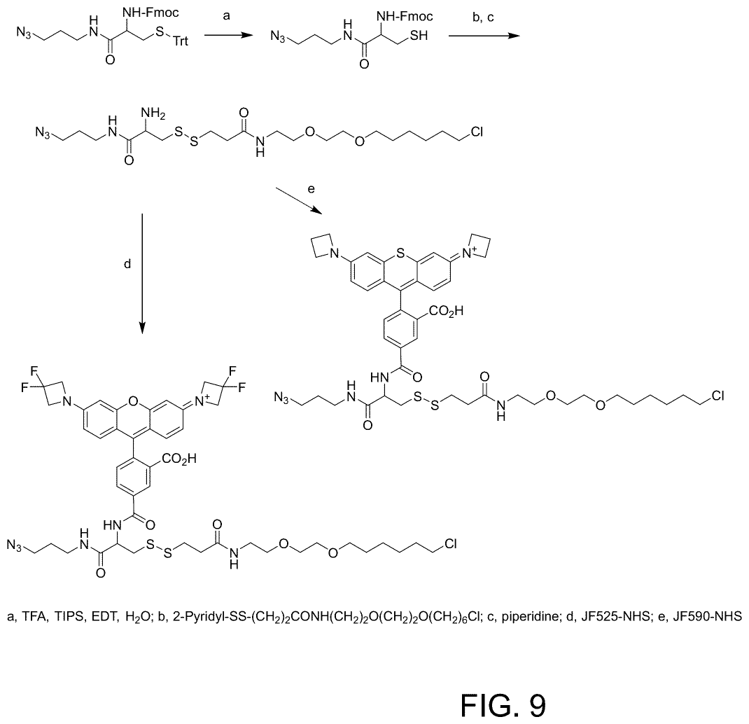

### Synthesis of JF525-PROMPT and JF570-PROMPT (see also FIG. 9)

### Synthesis of Fmoc-NH-cys(SH)—CO—NH—(CH₂)₃—N₃

Fmoc-NH-cys(S-Trt)-OH (59 mg, 100 μmol) and HATU (42 mg, 110 μmol) were dissolved in dry DMF (200 μL) in a plastic screw cap tube and 3-azidopropylamine (11 μL, 110 μmol, Click Chemistry Tools) followed by DIEA (38 μL, 220 μmol) were added with mixing. The reaction mix turned yellow and LC-MS revealed complete reaction in 30 mins; ES-MS (m/z) [M+Na]⁺ for C₄₀H₃₇N₅NaO₃S, 690.3; found 690.2. The reaction mixture was evaporated to yellow oil and trityl group removed by dissolving in TFA-H₂O-Triisopropylsilane-Ethanedithiol (92.5/2.5/2.5/2.5 v/v, 1 mL) and kept at room temperature for 2 h. Following evaporation to an oily solid, the desired product was purified by RP-HPLC, and lyophilized to a white solid. ES-MS (m/z) [M]⁺ for C₂₁H₂₄N₅O₃S, 426.2; found 426.1.

### Synthesis of NH₂-cys(S—S-2-(CH₂)₂CONH-HaloTag ligand)-CO—NH—(CH₂)₃—N₃

Fmoc-NH-cys(SH)—CO—NH—(CH₂)₃—N₃ (1.25 μmol, 50 μL of 25 mM solution in dry DMSO) was mixed with SPDP-HaloTag linker (1.25 μmol, 25 μL of 50 mM solution in dry DMSO) and NMM (2.5 μL, 25 μmol) added. LC-MS revealed complete reaction after 20 min, ES-MS (m/z) [M]⁺ for C₃₄H₄₈ClN₆O₆S₂, 735.3; found 735.3. Piperidine (20 μL) was added and the solution evaporated after 5 mins. The residue was dissolved in DMSO and HOAc (5 μL), product isolated by RP-HPLC, lyophilized and dissolved in dry DMSO (75 μL). ES-MS (m/z) [M]⁺ for C₁₉H₃₈ClN₆O₄S₂, 513.2; found 513.2.

### Synthesis of JF570-PROMPT: Janelia Fluor 570-CONH-cys(S—S—(CH₂)₂CONH-HaloTag ligand)-CO—NH—(CH₂)₃—N₃

The product from the previous step was reacted with JF570-NHS ester (0.4 μmol, 8 μl of 50 mM solution in dry DMSO) and NMM (10 μmol, 1 μl) for 2 days at room temp, the product isolated by RP-HPLC, lyophilized and dissolved in DMSO (100 μL) to give a 1.4 mM solution, measured by εₘₐₓ 100000 M⁻¹ cm⁻¹ in 0.1 M HCl in 95% ethanol at 574 nm. ES-MS (m/z) [M]⁺ for C₄₆H₅₈ClN₈O₇S₃, 965.3; found 965.3.

### Synthesis of JF525-PROMPT: Janelia Fluor 525-CONH-cys(S—S—(CH₂)₂CONH-HaloTag ligand)-CO—NH—(CH₂)₃—N₃

Prepared as for JF570-PROMPT. Measured by εₘₐₓ 80000 M⁻¹ cm⁻¹ in 0.1 M HCl in 95% ethanol at 532 nm. ES-MS (m/z) [M]⁺ for C₄₆H₅₄ClF₄N₈O₈S₂⁺, 1021.3; found 1021.3.

### Reaction with HaloTag Protein

PROMPT-525 or PROMPT-570 (0.5 μL of 1 mM solution in DMSO) were added to HaloTag protein (2.5 μL of 100 μM solution in PBS) diluted in 100 mM Na MOPS pH 7.2, and kept at room temperature for 2 h. Acetic acid (1 μL) was added and analyzed by LC-MS using PLRP-S 1000A column (8 μm, 50×2.1 mm, Agilent) eluting with linear 20-60% ACN-water with constant TFA (0.05%) in 16 min.

JF570-PROMPT: ES-MS (m/z) [M]⁺ for adduct with HaloTag protein (loss of HCl), C₄₆H₅₇N₈O₇S₃, (965.3-35.98) 929.3; found, deconvoluted masses, (HTP-JF570PROMPT:HTP), 35729.0-34798.6=930.4.

JF525-PROMPT: ES-MS (m/z) [M]⁺ for adduct with HaloTag protein (loss of HCl), C₄₆H₅₃F₄N₈O₈S₂, 985.3; found, deconvoluted masses, (HTP-JF525PROMPT:HTP), 35783.5-34798.6=984.9.

### Example 3. Validating the PS-PROMPT Method

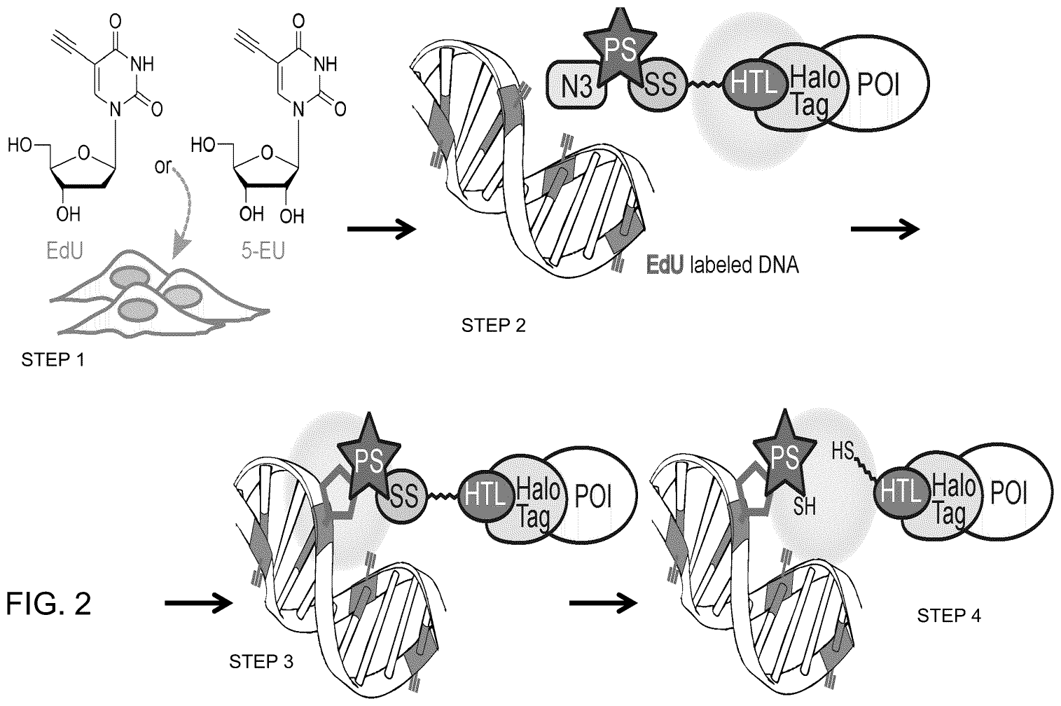

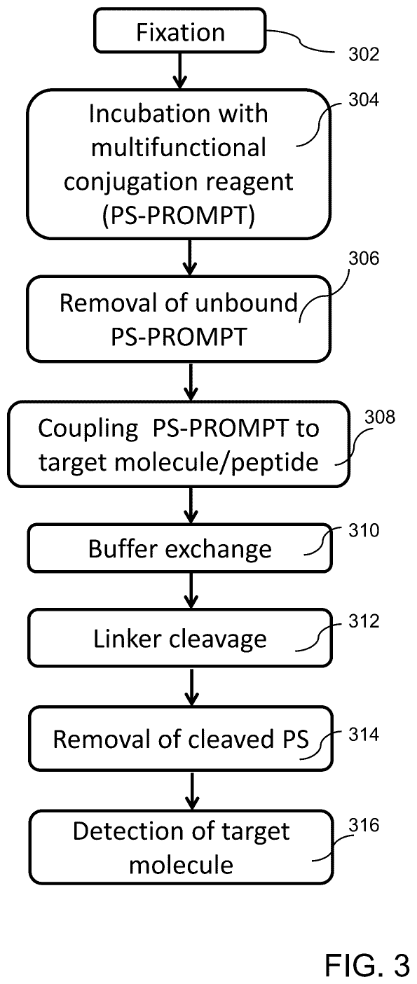

An exemplary design of a general labeling method disclosed herein (also called the PROMPT method) is shown in FIG. 3. A specific embodiment of the PROMPT method is shown in FIG. 2 and utilizes MCR shown in FIG. 1B. This MCR was designed to label polynucleotides in the vicinity of a protein of interest conjugated with a Halotag.

Referring to FIGS. 2 and 3, during initial step (step 1 (302)), live incorporation of the alkyne form of a metabolite of choice was done; Thymidine (5-Ethynyl-2′-deoxyuridine, EdU) was used for DNA labeling, or Uridine (5-Ethynyl-uridine, 5EU) was used for RNA labeling. Step 2 involved post-fixation labeling of protein of interest (POI) conjugated to Halotag using PS-PROMPT probe containing a Halotag ligand (HTL). Upon incubation (304), PS-PROMPT interacts with the HT-POI through its HTL module. Unbound PS-PROMPT is washed away (306). In step 3, PS-PROMPT immobilized to HT-POI is covalently linked to one nearby alkyne-nucleotide by click chemistry via its azido group (308) and the reaction buffer is removed (310). In step 4, the PS module is separated from the HTL module after the disulfide bond reduction (312). Unclicked PS can be washed away (314), while the clicked PS remains anchored to the nucleotide. The PS fluorescence observed via imaging (316) proves a successful transfer of the probe and therefore the relative vicinity between the protein and the incorporated nucleotide analog in the DNA or RNA strand.

After EdU or 5EU labeling, transfected cells were washed with culture medium and fixed with 2 minutes with room temperature fixative (4% EM grade paraformaldehyde (Electron Microscopy Sciences #19202)+0.1% EM grade glutaraldehyde (Ted Pella, #18426) in 0.1M Hepes (Sigma-Aldrich #H3375) pH7.4+2 mM CaCl2)) followed by a 1-hour incubation with 4C fixative. Next, the cells were washed three times with PBS and incubated for 10 minutes with PBS+10 mM glycine. The cells were then incubated for 30 minutes with 200 nM of PS-PROMPT probe in PBS+0.1% BSA (Sigma-Aldrich #A8022)+0.1% saponin (Sigma-Aldrich #S4521), then washed five times with PBS+0.1% BSA+0.1% saponin, and five times with PBS. For the click chemistry step, the cells were incubated for 2 times 30 minutes with 50 mM Hepes pH7.4+100 mM NaCl+2 mM CuSO4+10 mM of sodium ascorbate (Sigma-Aldrich #A7631). The reaction was terminated with ten washes with PBS. For the disulfide bond reduction, cells were incubated three times 10 minutes with freshly prepared 10 mM DTT (Sigma-Aldrich #D9779) in PBS. Released photosensitizers were washed away with five washes with PBS+0.1% BSA+0.1% saponin and five washes with PBS.

To evaluate the ability of TMR-PROMPT to bind to HaloTag, the histone H2B fused with Halotag was expressed in U2OS cells, and the cells were incubated with TMR-Halotag ligand (TMR-HTL) or TMR-PROMPT. TMR-HTL was bound efficiently to Halotag-H2B whether the incubation was on live cells or following aldehyde fixation, whereas TMR-PROMPT labeled its target only when added after fixation. To avoid premature cleavage of disulfide group by intracellular glutathione, the PROMPT method was performed after aldehyde fixation. Fixed U2OS cells transfected with Halotag-H2B were incubated with TMR-HTL or TMR-PROMPT, then treated with 10 mM DTT and heavily washed. With or without DTT, cells incubated with TMR-HTL displayed a constant TMR fluorescent signal, verifying that DTT does not affect the fluorescent properties of TMR. Last, functionality of the azido group of TMR-PROMPT was verified by performing click chemistry with TMR-PROMPT on EdU treated cells. Only when cells were labeled with EdU, was TMR fluorescence observed in the nucleus.

Next, the feasibility of the PROMPT method was tested using as a model system the interaction between the histone H2B with DNA. For CLEM, JF570-PROMPT was used, as JF570 is a more efficient photosensitizer than TMR. JF570-PROMPT was synthesized as described above. Reaction of JF570-PROMPT with purified HaloTag protein in vitro was confirmed by LC-MS, giving the expected mass for the labeled protein.

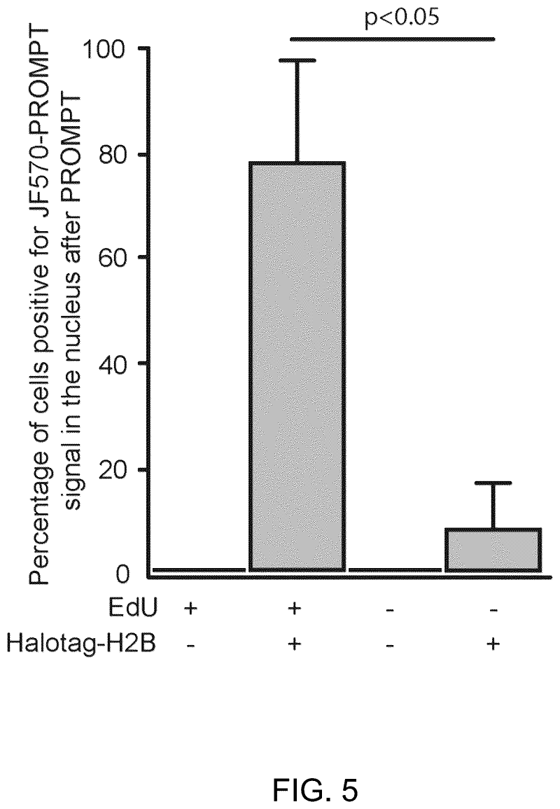

For the JF570-PROMPT method (see steps in FIG. 2 and FIG. 3), following overnight incubation with 10 uM EdU, cells transfected with Halotag-H2B were fixed with 4% paraformaldehyde (PFA) and 0.1% glutaraldehyde (GA) and briefly incubated with JF570-PROMPT in the presence of saponin to facilitate probe penetration. After washing, it was verified that only Halotag-H2B expressing cells displayed JF570 fluorescence in the nucleus, demonstrating the covalent bond formation between JF570-PROMPT and Halotag-H2B. Next, Cu(I)-catalyzed azide-alkyne cyclization (CuAAC) click chemistry was performed to trigger the binding of the JF570-PROMPT immobilized on Halotag-H2B to the nearest clickable substrate, an alkyne-uridinyl residue incorporated into DNA. After washing to remove the click chemistry solution and terminate the reaction, the cells were treated with DTT to cleave the disulfide link between JF570 and Halotag-H2B. After intense washing to remove unclicked but cleaved JF570-PROMPT, the JF570 fluorescence was imaged. It was shown that only under the condition where Halotag-H2B was expressed in EdU-treated cells did the majority of cells displayed an above background JF570 fluorescent signal in the nucleus (FIG. 5).

### Example 4. Visualizing Histone H2B-DNA Interaction in U2OS Cells with PROMPT and CLEM

In this example, photooxidation of DAB through the illumination of the remaining JF570 after the PROMPT method in cells expressing Halotag-H2B and labeled with EdU was performed. After osmification, embedding, and sectioning, the overall darkening of the correlated nucleus by electron microscopy was verified. At higher magnification, a high concentration of puncta, which is more opaque to electrons, was visualized.

### Example 5. PROMPT Dependence on Molecular Distances in Cellular Ultrastructure

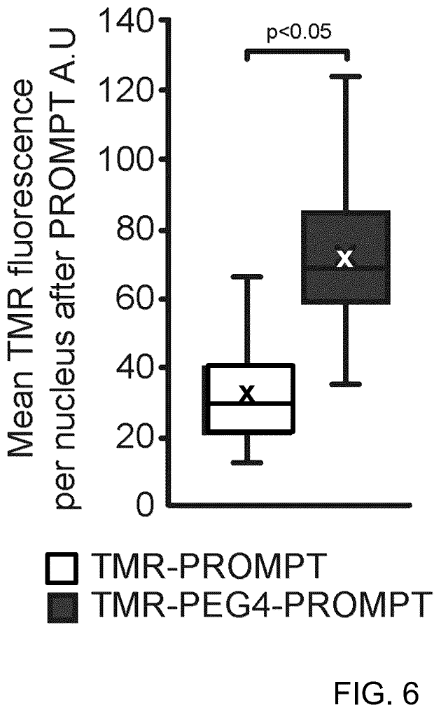

Considering the structural constraints on the PS (detectable label) transfer success rate, it was reasoned that elongating the linker chain of the PS-PROMPT would increase its efficiency. Referring to FIG. 6, the effect of adding a PEG4 linker to TMR-PROMPT on the TMR transfer efficiency was examined. U2OS cells expressing Halotag-H2B and pretreated with 5 uM EdU were processed with PROMPT using TMR-PROMPT probe or TMR-PEG4-PROMPT. The higher fluorescence signal is observed by confocal for TMR-PEG4-PROMPT compared to TMR-PROMPT. Error bars are box-and-whiskers plots containing the mean (X), quartiles (box), and minimum and maximum observations (whiskers). In cells expressing Halotag-H2B and labeled with EdU, the TMR fluorescence intensity is significantly greater after PROMPT when using TMR-PEG4-PROMPT. Elongating the probe not only compensates for unfavorable orientations of partners but also increases the radius by which the PS-PROMPT can find a clickable target.

### Example 6. Visualizing Fibrillarin-RNA Interaction in U2Os Cells with PS-PROMPT and CLEM

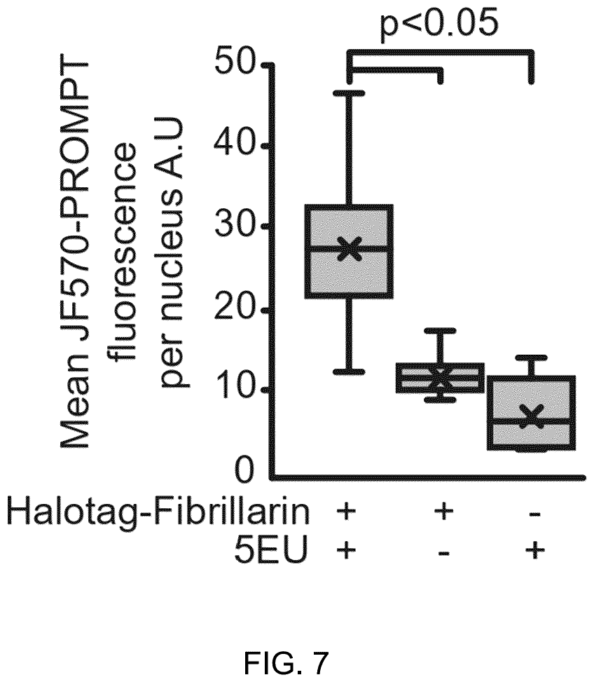

As the feasibility of PROMPT to pinpoint protein-DNA binding partners by CLEM was demonstrated, replacing EdU with 5-Ethinyl Uridine (5EU) should make PROMPT amendable to the study of protein-RNA interaction. As a study system, the PROMPT method was tested on the interaction of fibrillarin with RNAs. As a component of the C/D box small nucleolar ribonucleoproteins, fibrillarin is involved in the 2′-O-methylation of rRNAs. U2OS cells transfected with or without the Halotag-Fibrillarin construct, pretreated overnight with or without 1 mM 5EU, were processed with the PROMPT method. Referring to FIG. 7, after PROMPT using JF570-PROMPT, the JF570 fluorescent signal was only detected in cells treated with both Halotag-Fibrillarin and 5EU. Quantification of the mean JF570-PROMPT fluorescence signal per nucleus in the treated cells is shown. Error bars are box-and-whiskers plots containing the mean (X), quartiles (box), and minimum and maximum observations (whiskers). Similar observations were made using JF525-PROMPT where JF570 has been replaced with the yellow photosensitizer, JF525. Completing the protocol with DAB photooxidation by JF570, the extent of DAB polymerization was dependent on the JF570 fluorescence intensity as expected. The 3D distribution of the fibrillarin-RNA complexes was observed throughout the nucleus. In summary, the PROMPT method confirmed the known interaction of fibrillarin with rRNAs for their methylation and revealed potential functions in the nucleoplasm.

### Example 7. Extensions of the PROMPT Method

A few variants of MCR and the PROMPT method were disclosed in foregoing Examples 1-6. As will be readily apparent from these examples, the PROMPT method can be extended beyond the disclosed designs.

The PROMPT method is relatively simple and, except for the MCR, does not require specialized chemicals or equipment. It can potentially be applied to visualize the interaction of any fusion protein with cellular components that can incorporate clickable metabolites such as proteins, nucleotides, sugars, lipids, or enzymatic inhibitors. As clickable metabolites are fed to live cells, the method is amenable for spatial-temporal studies using pulse-chase to detect lifetimes of fusion proteins interaction with newly synthesized macromolecules.

The choice of the protein fused to a tag could facilitate the identification and subcellular localization of specific polynucleotide sequences in cells. For example, in combination with dCas9-Halotag with a specific guide RNA (Chen, B. et al. Dynamic Imaging of Genomic Loci in Living Human Cells by an Optimized CRISPR/Cas System. Cell 155, (2013)), one can target only the binding fraction of dCas9 to the chosen DNA sequences. While relatively weak, the signal should be detectable by removal of the excess, unbound fraction of labeled dCas9-Halotag that loses fluorescence by the PROMPT method.

The MCR is composed of different functional modules, and the modification of one or several modules broadens the range of potential applications of the disclosed method. For example, replacement of the Halotag ligand module by SNAP or CLIP ligands (benzyl guanine and benzyl cytosine) (disclosed in Gautier, A. et al. An Engineered Protein Tag for Multiprotein Labeling in Living Cells. Chem. Biol. 15, (2008)) expands the spectrum of protein targeting systems and enables simultaneous PROMPT for multiple proteins. In addition, the important applications of the PROMPT method may come from the replacement of the detectable label. For instance, the photosensitizer can be replaced by a wide range of fluorophores, including the ones compatible with super-resolution, such as JF549 or JF646 (disclosed in Grimm, J. B. et al., A general method to improve fluorophores for live-cell and single-molecule microscopy. Nat. Methods 12, (2015)). An exemplary modification would be to place two fluorophores (of non-overlapping spectral properties or part of a FRET donor-acceptor couple) on each side of the selectively cleavable linkage (e.g., disulfide bond). In this way, one can study the total protein pool with the fluorophore on the Halotag ligand side (based on the structure of MCR shown in FIG. 1B) and the fraction interacting with the metabolite of choice by looking at the fluorophore on the clickable side of the MCR. In addition, other very promising uses of the disclosed method may emerge from the exchange of the fluorophore for (or addition of) a biotin group, therefore making the PROMPT method amendable to polypeptide pulldown for sequencing and/or mass spectrometry. In combination with MCRs having variable carbon chain length of the cleavable linker, one can start to map in 3D the relative distance of identified sequences of DNA, RNA, protein, metabolites, to the biotin donor (the protein of interest).

### Example 8. Additional Variants and Applications of the PROMPT Method

In this example, variants of target molecules suitable for the disclosed methods and modified to have a first complementary bioorthogonal reactive handle are provided. Different types of the modified target molecules that can be examined by the disclosed methods are listed. It is understood that different reactive handles known in the art, including bioorthogonal reactive handles, can be employed to generated modified target molecules disclosed in this and other Examples.

Nucleic Acids as Target Molecules.

The following modified nucleotides can be incorporated into target polynucleotides: 5-Ethynyl Uridine (5-EU): an alkyne-containing uridine analogue for RNA labeling; 5-Ethyl-2′-deoxyuridine (EdU): an alkyne-containing thymidine analogue for DNA labeling.

Using the disclosed methods, one can identify the sequence targeted by a given transcriptional factor or the identity of the non-coding RNAs interacting with a protein of interest. For instance, the protein STAT3 serves as a critical transcription factor for the regulation inflammation and tissue repair, and for which the over-activation facilitates tumorigenesis. With the disclosed methods, one can estimate the fraction of STAT3 bound to its genomic target in macrophage and identify different population of macrophages in tissue, some inhibiting, and some facilitating cancer proliferation.

Carbohydrates as Target Molecules.

The following modified sugar monomers can be incorporated into target carbohydrates: O-Alkyne-Trehalose (Alkyne-modified, non-mammalian disaccharide precursor essential for mycomembrane).

Trehalose is abundant and widely distributed in nature, as it occurs in bacteria, yeast, fungi, plants, and invertebrates. Insects store high levels of blood trehalose, which can be rapidly utilized to permit flight. In plants, trehalose's biosynthetic precursor, trehalose-6-phosphate, has been implicated in plant growth regulation and development. Some actinobacteria express lentztrehalose A, a molecule resistant to degradation. Trehalose is also essential for growth and virulence of globally significant pathogens such as Mycobacterium tuberculosis, which not only uses trehalose for energy storage and stress resistance, but also incorporates trehalose into virulence-associated cell envelope glycolipids.

The following modified sugar can also be incorporated into target carbohydrates: N-(4-pentynoyl)-mannosamine-tetraacylated(Ac4ManNAl) (unnatural, alkyne-containing tetraacylated monosaccharide building block; can be incorporated into sialic acids).

Sialic acids, a family of monosaccharides widely distributed in higher eukaryotes and certain bacteria, are determinants of many functional glycans that play central roles in numerous physiological and pathological processes. For example, the sialic acid-containing epitope Siaα2-6Gal serves as the cellular receptor for human influenza-A and -B viruses during infection, and linear homopolymers of sialic acids, known as polysialic acid (PSA), modulate neuronal synapse formation in mammalian development. The expression of sialoglycoconjugates, such as sialyl Lewis x, sialyl Tn (STn), and PSA, is also a common feature shared by numerous cancers.

The following modified sugar can also be incorporated into target carbohydrates: N-(4-pentynoyl)-glucosamine-tetraacylated(Ac4GlcNAl) (unnatural, alkyne-containing tetraacylated monosaccharide building block).

Protein O-GlcNAcylation is a specific form of protein glycosylation involving the addition of a single N-acetylglucosamine (GlcNAc) moiety to serine and threonine residues. While the biological role of O-GlcNAcylation remains less understood compared to other cell signaling post translational modifications (PTMs) such as phosphorylation, thousands of 0-GlcNAc substrates have been identified. O-GlcNAcylation is a ubiquitous PTM implicated in various aspects of cellular functions, including gene transcription, cell signaling, and stress response. In particular, 0-GlcNAc is well-known to play a cytoprotective function in response to various forms of stress. Importantly, 0-GlcNAcylation lies at the crossroads of nutrient signaling and cellular stress response. Deregulation of O-GlcNAcylation has been linked to many diseases including diabetes, neurodegenerative diseases and cancer.

The following modified sugar can also be incorporated into target carbohydrates: N-(4-pentynoyl)-galactosamine-tetraacylated(Ac4GalNAl) (unnatural, alkyne-containing tetraacylated monosaccharide building block).

O-Linked N-acetylgalactosamine modification in an alpha linkage to the hydroxyl of serine and threonine residues is often referred to as mucin-type O-glycosylation, as the mucins are heavily 0-GalNAc modified. In the mucins, literally hundreds of sites on the polypeptide can be decorated with a variety of O-GalNAc-initiated extended core structures. O-GalNAc-initiated glycoproteins appear to play a variety of essential roles. Among these is the ability of the mucins to hydrate and protect tissues by trapping bacteria. These O-glycans can also significantly alter the conformation of the protein and on the heavily modified proteins may protect the polypeptide from proteolytic digestion. O-GalNAc structures also appear to play an essential role in sperm-egg interactions. From a pathophysiological perspective, 0-GalNAc modification appears to play a critical role in the immune system, cell-cell interactions, and cancer.

The disclosed methods would allow to label carbohydrates listed above using appropriate MCRs and proteins that are located in proximity from the carbohydrates. The disclosed methods would facilitate the comprehension of the role of various glycosylation modifications by highlighting interactions with selected proteins, but also may reveal the glycosylated fraction of a protein of interest. As an example of STAT3, it was previously shown that O-GlcNAcylation of STAT3 negatively regulates STAT3 phosphorylation and reduces IL-10 production. Looking at the O-GlcNAcylation of STAT3 will provide a direct readout of inflammation in situ, and of effect on tumor.

Newly Synthesized Proteins as Target Molecules.

Use of the inventive PROMPT for protein-protein interactions expands many possible applications similar to the ways in which FRET is now widely used. Alkyne groups can be incorporated into specific target proteins using a protein tag such as HaloTag, SNAP-tag, CLIP-tag, tetracysteine, and labeling with an alkyne-containing ligand for the tag used. As will be apparent to those of skill in the art, a protein tag that is used in such applications would be different and orthogonal to that used to bind the PROMPT probe to the polypeptide of interest (POI).

Examples of suitable alkyne ligands for HaloTag include propargyl-chloroalkanes. SNAP-tag examples include benzylguanine-alkynes. CLIP-tag examples include benzylcytosine-alkynes. Tetracysteine examples include biarsenical-alkynes. PEG linkers of variable length may be used between the alkyne and ligand.

The following moiety can be incorporated into target polypeptides: O-propargyl-puromycine (OPP) (an alkyne analog of puromycin that is incorporated into newly translated proteins in complete methionine-containing media).

The use of this analog is pertinent for understanding protein synthesis, folding and turnover. For example, one could look at the binding of the chaperone BIP (binding immunoglobulin protein) to newly synthesized proteins. This would indicate the level of unfolded proteins in response to cellular stress, which can be linked to neurodegenerative disease such as Alzheimer's disease.

In some embodiments, other moieties may be incorporated into newly synthesized target proteins, such as unnatural amino acid residues or azide/alkyne amino acid residues (e.g., propargyl glycine, alkynehomoalanine).

Fatty Acids as Target Molecules.

The following modified moieties can be incorporated into target fatty acid molecules: alkynyl myristic acid—an analog of 14 carbon saturated fatty acid; alkynyl palmitic acid—an analog of 16 carbon saturated fatty acid; alkynyl stearic acid—an analog of 18 carbon saturated fatty acid; alkynyl cholesterol—modified cholesterol with an omega-terminal alkyne; alkynyl sphinganine
a modified precursor of ceramides and sphingolipids.

This panel of lipid analogs in association with the disclosed methods is useful for clarifying the interplay of proteins with lipids, in processes such as energy metabolism, membrane biogenesis, intra cellular traffic, and lipid dependent signaling.

For instance, one could look at the in vivo interaction of cholesterol with NPC2, a cholesterol binding protein, related to the neurodegenerative disease Niemann-Pick type C2.

Polyketides as Target Molecules.

Alkyne-tagged polyketide synthesis method is disclosed in Porterfield WB, Poenateetai N, Zhang W. Engineered Biosynthesis of Alkyne-Tagged Polyketides by Type I PKSs. iScience. 2020 Mar. 27; 23(3):100938. Alkyne-modified polyketides can be used in the disclosed methods. Alkyne-modified polyketides are analogs of many compounds with high bioactivity, such as environmental toxins (e.g., aflatoxin), antibiotics (e.g., erythromycin and tetracycline), antineoplastics (e.g., daunorubicin) and immunosuppressants (e.g., rapamycin).

Erythromycin and tetracycline are inhibitors of the translation in bacteria. The disclosed methods can be used to study the efficiency of targeting of these antibiotics to the ribosome in situ and might highlight new mechanisms of antibiotic resistance.

The disclosed methods can also be used to explore the interaction/proximity of a protein of interest with any clickable biomolecule in situ.

In another example, the replacement of the fluorophore-based detectable label by a biotin-based label would enable selective precipitation of the neighboring/interacting biomolecules and their precise identification by analytical methods, such as DNA/RNA sequencing methods or mass spectrometry.

### OTHER EMBODIMENTS

The foregoing detailed description is provided to aid those skilled in the art in practicing the present invention. However, the invention described and claimed herein is not to be limited in scope by the specific embodiments herein disclosed because these embodiments are intended as illustration of several aspects of the invention. Any equivalent embodiments are intended to be within the scope of this invention. Indeed, various modifications of the invention in addition to those shown and described herein will become apparent to those skilled in the art from the foregoing description which do not depart from the spirit or scope of the present inventive discovery. Such modifications are also intended to fall within the scope of the appended claims.

### REFERENCES CITED

All publications, patents, patent applications and other references cited in this disclosure are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present invention.

What is claimed is:

1. A multifunctional conjugation reagent, comprising:
a first bioorthogonal reactive handle configured to be attached to a target molecule or to a modified target molecule;
a detectable label;
a second reactive handle configured to be attached to a polypeptide or to a modified polypeptide; and
a linker disposed between the second reactive handle and the detectable label, wherein the linker comprises a selectively cleavable linkage.

2. The conjugation reagent of claim 1, wherein the first bioorthogonal reactive handle is selected from the group consisting of: azide, tetrazine, methyltetrazine, cyclopropene, trans-cyclooctene, substituted trans-cyclooctene, alkene, terminal alkyne, cyclooctyne tetrazine, ester, thioester, nitrile, alkylating agent, phosphate ester, azidoacetamide, semicarbazide, phospholipid, ketone, aldehyde, hydrazide, alkoxyamine, phosphine, nitrone, nitrile oxide, diazo compound, tetrazole, quadrocyclane, iodobenzene, cyclooctyne, bicyclononyne (BCN), diarylcyclooctyne (DBCO), norbornene, vinyl, isonitrile, and cycloaddition reactant.

3. The conjugation reagent of claim 1, wherein the second reactive handle is a bioorthogonal reactive handle.

4. The conjugation reagent of claim 3, wherein the target molecule is a first protein and the polypeptide is a second protein.

5. The conjugation reagent of claim 1, wherein the second reactive handle is selected from the group consisting of: a Halotag ligand, a SNAP ligand, a CLIP ligand, tetracysteine ligand, and a THP-ligand.

6. The conjugation reagent of claim 1, wherein the detectable label comprises a fluorogenic moiety or a photosensitizer.

7. The conjugation reagent of claim 1, wherein the detectable label comprises biotin or a biotin derivative.

8. The conjugation reagent of claim 1, wherein the selectively cleavable linkage is selected from the group consisting of: a disulfide cleavable by reduction, a photolabile linkage or vicinal diol-containing linkage cleavable by periodate, and protease cleavable linkage.

9. The conjugation reagent of claim 1, wherein the target molecule is a biological macromolecule selected from the group consisting of: a polynucleotide, a lipid, a target polypeptide, and a carbohydrate.

10. The conjugation reagent of claim 1, wherein the linker further comprises a poly(ethylene glycol) PEG polymer, a poly(ethylene oxide) PEO polymer, a polymethylene, or a peptide.

11. The conjugation reagent of claim 1, wherein the first bioorthogonal reactive handle and the second reactive handle are located at different termini of the conjugation reagent.

12. A method for labeling a target molecule that is located in proximity to a polypeptide, the method comprising the steps of:
(a) providing the target molecule having a first complementary bioorthogonal reactive handle configured to react with a first bioorthogonal reactive handle of a multifunctional conjugation reagent, wherein the multifunctional conjugation reagent comprises the first bioorthogonal reactive handle, a detectable label, a second reactive handle configured to be attached to the polypeptide, and a linker located between the second reactive handle and the detectable label, wherein the linker comprises a selectively cleavable linkage;
(b) contacting the polypeptide with the multifunctional conjugation reagent, thereby generating the polypeptide comprising the first bioorthogonal reactive handle and detectable label;
(c) providing conditions for reaction between the first complementary bioorthogonal reactive handle of the target molecule and the first bioorthogonal reactive handle of the polypeptide; and
(d) providing conditions for cleavage of the selectively cleavable linkage, wherein after the cleavage the detectable label remains attached to the target molecule.

13. The method of claim 12, wherein the target molecule specifically binds to the polypeptide.

14. The method of claim 12, which is for labeling the target molecule located inside a cell, wherein labeling of the target molecule at step a) occurs within the cell, and the target molecule are provided in proximity to the polypeptide at step b) within the cell.

15. The method of claim 14, further comprising providing a fixation solution to the cell after step b) and before step c).

16. The method of claim 12, wherein the first bioorthogonal reactive handle is selected from the group consisting of: azide, tetrazine, methyltetrazine, cyclopropene, trans-cyclooctene, substituted trans-cyclooctene, alkene, terminal alkyne, cyclooctyne tetrazine, ester, thioester, nitrile, alkylating agent, phosphate ester, azidoacetamide, semicarbazide, phospholipid, ketone, aldehyde, hydrazide, alkoxyamine, phosphine, nitrone, nitrile oxide, diazo compound, tetrazole, quadrocyclane, iodobenzene, cyclooctyne, bicyclononyne (BCN), diarylcyclooctyne (DBCO), norbornene, vinyl, isonitrile, and cycloaddition reactant.

17. The method of claim 12, wherein the second reactive handle is a bioorthogonal reactive handle.

18. The method of claim 17, wherein the target molecule is a first protein and the polypeptide is a second protein.

19. The method of claim 12, wherein the second reactive handle is selected from the group consisting of: a Halotag ligand, a SNAP ligand, a CLIP ligand, tetracysteine ligand, and a THP-ligand.

20. The method of claim 12, wherein the detectable label comprises a fluorogenic moiety or a photosensitizer.

21. The method of claim 12, wherein the detectable label comprises biotin or a biotin derivative.

22. The method of claim 12, wherein the selectively cleavable linkage is selected from the group consisting of: a disulfide cleavable by reduction, a photolabile linkage or vicinal diol-containing linkage cleavable by periodate, and protease cleavable linkage.

23. The method of claim 12, wherein the target molecule is a biological macromolecule selected from the group consisting of: a polynucleotide, a lipid, a target polypeptide, and a carbohydrate.

24. The method of claim 12, wherein the linker further comprises a poly(ethylene glycol) PEG polymer, a poly(ethylene oxide) PEO polymer, a polymethylene, or a peptide.

25. The method of claim 12, wherein the first bioorthogonal reactive handle and the second reactive handle are located at different termini of the conjugation reagent.
